(19) United States
(12) Patent Application Publication
(10) Pub. No.: US 2017/0259088 A1
(43) Pub. Date: Sep. 14, 2017

(54) RESPIRATORY DEVICE FOR HEAT STRESS, HEAT STROKE AND HYPOTHERMIA MITIGATION

(71) Applicants: Nicholas G. Garafolo, Akron, OH (US); Andrew P. Killmeyer, Medina, OH (US); Brandon L. Null, Spencer, OH (US); Marisa A. Seeley, Medina, OH (US); Daniel R. Slattery, Akron, OH (US); Nick J. Tsangeos, Canton, OH (US); Jeffrey J. Quartermaine, Sandusky, OH (US); Harry J. Harris, Akron, OH (US); Gopal Nadkarni, North Canton, OH (US)

(72) Inventors: Nicholas G. Garafolo, Akron, OH (US); Andrew P. Killmeyer, Medina, OH (US); Brandon L. Null, Spencer, OH (US); Marisa A. Seeley, Medina, OH (US); Daniel R. Slattery, Akron, OH (US); Nick J. Tsangeos, Canton, OH (US); Jeffrey J. Quartermaine, Sandusky, OH (US); Harry J. Harris, Akron, OH (US); Gopal Nadkarni, North Canton, OH (US)

(21) Appl. No.: 15/456,794

(22) Filed: Mar. 13, 2017

Related U.S. Application Data
(60) Provisional application No. 62/306,740, filed on Mar. 11, 2016.

(51) Int. Cl. A62B 9/00; A61M 16/10; A61M 16/06; A62B 18/04; A61F 7/00

(52) U.S. Cl. CPC A62B 9/003; A62B 18/045; A61F 7/0085; A61M 16/06; A61M 16/109; A61F 2007/0061; A61F 2007/0063; A61F 2007/0075; A61M 2205/3606; A61M 2205/362; A61M 2205/3673

(57) ABSTRACT

A respiration-based body temperature modifying apparatus including an air temperature modifying element producing temperature-altered air and a respiration zone that, in use, communicates with the respiratory system of the user and receives the temperature-altered air so that the user can draw the temperature-altered air from the respiration zone into the lungs to alter body temperature.

### CROSS-REFERENCE TO RELATED APPLICATIONS

The present application claims priority to U.S. Provisional Patent Application Ser. No. 62/306,740 filed Mar. 11, 2016, the entirety of which is incorporated herein by reference.

### STATEMENT REGARDING FEDERALLY SPONSORED RESEARCH OR DEVELOPMENT

This invention was made with government support under FA8650-14-C-7400 awarded by The USAF Air Force Research Laboratory. The government has certain rights in the invention.

### FIELD OF THE INVENTION

The present invention generally relates to a respiration-based body temperature modifying apparatus. In some embodiments, the present invention is directed toward a respiration-based body temperature modifying apparatus comprising an air temperature modifying element and a respiration zone that communicates with the respiratory system of the user. In some embodiments, the respiration-based body temperature modifying apparatus comprises an inlet zone defined where ambient air proximate the air temperature modifying element is affected by the air temperature modifying element to produce the temperature-altered air.

### BACKGROUND OF THE INVENTION

There is a need to safely, rapidly, and effectively maintaining body core temperature in extreme environments (whether hot or cold) to mitigate temperature related illnesses (e.g., heat stress, heat stroke, hypothermia). On a more simplistic level, there is always a desire to maintain a comfortable body temperature in any given hot or cold environment, whether or not such environment could lead to illness or anything serious beyond discomfort.

Wearing of heavy protective equipment exasperates the problems associated with temperature related discomfort or illness. Examples include motorcycle and ATV helmets, protective equipment for steel works, military operation, and protective equipment for surgeons and hazmat operators. Operating in extreme environments contributes to the problem. Other factors that inhibit the dissipation of thermal energy away from the body include: radiation from equipment, surfaces, and vehicles, and heat trapped in vehicles, tents, and other poorly ventilated spaces.

Various high-tech clothing has been provided in the art to address this need to remain comfortable and/or safe in various environments. Waterproof and breathable fabrics help retain body heat while allowing for the evaporation of sweat. Down, wool, and various synthetic fabrics or fill material are provided in clothing to keep the body warm, while other clothing fabrics are designed to keep the body cool. Clothing with heating and/or cooling elements are also of interest and have been and continue to be developed. However, addressing body temperature moderation through such clothing is not adequate, and focuses unnecessarily on the body's exterior (i.e. covering the exterior of the body with clothing suitable for maintaining body heat (to heat the body) or dissipating body heat (to cool the body).

The present invention addresses the need in the art for apparatus to safely, rapidly, and effectively remove undesired heat away from the body, to cool it, or add heat to the body, to heat it. The present invention does so by focusing on advantageous use of the body's respiration function.

### SUMMARY OF THE INVENTION

In a first embodiment, the present invention provides a respiration-based body temperature modifying apparatus comprising an air temperature modifying element producing temperature-altered air; and a respiration zone that, in use, communicates with the respiratory system of the user and receives the temperature-altered air so that the user can draw the temperature-altered air from the respiration zone into the lungs to alter body temperature.

In a second embodiment, the present invention provides a respiration-based body temperature modifying apparatus as in any of the above embodiments, wherein the respiration-based body temperature modifying apparatus further includes an inlet zone defined where ambient air proximate the air temperature modifying element is affected by the air temperature modifying element to produce the temperature-altered air.

In a third embodiment, the present invention provides a respiration-based body temperature modifying apparatus as in any of the above embodiments, further comprising an inlet chamber, wherein the air temperature modifying element and the inlet zone reside in the inlet chamber; and an inlet passage for the passage of external air into the inlet chamber.

In a fourth embodiment, the present invention provides a respiration-based body temperature modifying apparatus as in any of above embodiments, further comprising a respiration chamber, the respiration zone residing in the respiration chamber; and a inhalation passage from the inlet chamber to the respiration chamber for the passage of temperature-altered air from the inlet chamber to the respiration chamber.

In a fifth embodiment, the present invention provides a respiration-based body temperature modifying apparatus as in any of the above embodiments, wherein the respiration chamber, in use, seals about the user's face to cover a mouth or nose or both the mouth and nose of the user.

In a sixth embodiment, the present invention provides a respiration-based body temperature modifying apparatus as in any of the above embodiments, further comprising an outlet in the respiration chamber for exhausting exhaled breath of the user to the exterior atmosphere.

In a seventh embodiment, the present invention provides a respiration-based body temperature modifying apparatus as in any of the above embodiments, wherein the air temperature modifying element is a ceramic strip heater.

In an eighth embodiment, the present invention provides a respiration-based body temperature modifying apparatus as in any of the above embodiments, further comprising an outlet chamber; and an outlet in the outlet chamber for exhausting exhaled breath of the user to the exterior atmosphere, the respiration chamber communicating with the outlet chamber through an exhalation passage.

In a ninth embodiment, the present invention provides a respiration-based body temperature modifying apparatus as in any of the above embodiments, wherein the air temperature modifying element is selected from hot side heating elements and cold side cooling elements provided by a thermoelectric device having both the hot side heating elements and the cold side cooling elements, such that one of the hot side heating elements and the cold side cooling elements reside in the inlet chamber and serve as the air-temperature modifying element, and the other of the hot side heating elements and the cold side cooling elements reside in the outlet chamber, wherein exhaled breath of the user passes over the hot side heating or cold side cooling element residing in the outlet chamber.

In a tenth embodiment, the present invention provides a respiration-based body temperature modifying apparatus as in any of the above embodiments, further comprising a one-way inhalation valve in the inhalation passage allowing for the passage of temperature-altered air from the inlet chamber to the respiration chamber.

In an eleventh embodiment, the present invention provides a respiration-based body temperature modifying apparatus as in any of the above embodiments, wherein the one-way inhalation valve is chosen such that the user's respiration is sufficient to draw temperature-altered air through the one-way inhalation valve.

In a twelfth embodiment, the present invention provides a respiration-based body temperature modifying apparatus as in any of the above embodiments, further comprising an inlet valve in the inlet passage, wherein the inlet valve is chosen such that the user's respiration is sufficient to draw external air through the inlet valve and into the inlet chamber.

In a thirteenth embodiment, the present invention provides a respiration-based body temperature modifying apparatus as in any of the above embodiments, further comprising a one-way exhalation valve in the exhalation passage or in the outlet chamber, wherein the one-way exhalation valve is chosen such that the user's respiration is sufficient to exhaust exhaled air through the one-way exhalation valve.

In a fourteenth embodiment, the present invention provides a respiration-based body temperature modifying apparatus as in any of the above embodiments, wherein the hot side heating elements and the cold side cooling elements can be selectively switched to switch the positioning of the hot side heating elements and the cold side cooling elements in the inlet chamber and the outlet chamber, thus switching the type of air, be it heated or cooled, at the inlet zone.

In a fifteenth embodiment, the present invention provides a respiration-based body temperature modifying apparatus as in any of the above embodiments, the thermoelectric device is a Peltier chip including a plurality of heat transfer fins serving as the cold side cooling elements and a plurality of heat transfer fins serving as the hot side heating elements.

In a sixteenth embodiment, the present invention provides a respiration-based body temperature modifying apparatus as in any of the above embodiments, further comprising a helmet, wherein the respiration zone is provided in the helmet.

In a seventeenth embodiment, the present invention provides a respiration-based body temperature modifying apparatus as in any of the above embodiments, wherein the air temperature modifying element is selected from hot side heating elements and cold side cooling elements provided by a thermoelectric device having both the hot side heating elements and the cold side cooling elements, such that one of hot side heating elements and the cold side cooling elements reside in the respiration zone and serve as the air-temperature modifying element.

In an eighteenth embodiment, the present invention provides a respiration-based body temperature modifying apparatus as in any of the above embodiments, where the other of the hot side heating elements and the cold side cooling elements reside exteriorly of the helmet.

In a nineteenth embodiment, the present invention provides a respiration-based body temperature modifying apparatus as in any of the above embodiments, further comprising a fan moving air across the other of the hot side heating elements or cold side cooling elements residing exteriorly of the helmet.

In a twentieth embodiment, the present invention provides a respiration-based body temperature modifying apparatus as in any of the above embodiments, further comprising a respiration side fan moving air across the hot side heating or cold side cooling elements residing in the respiration zone.

In a twenty-first embodiment, the present invention provides a respiration-based body temperature modifying apparatus as in any of the above embodiments, wherein the hot side heating elements and the cold side cooling elements can be selectively switched to switch the positioning of the hot side heating elements and the cold side cooling elements in the respiration zone and exteriorly of the helmet.

In a twenty-second embodiment, the present invention provides a respiration-based body temperature modifying apparatus as in any of the above embodiments, wherein the air temperature modifying element is a Peltier chip including a plurality of heat transfer fins serving as the cold side cooling elements and a plurality of heat transfer fins serving as the hot side heating elements.

In a twenty-third embodiment, the present invention provides a respiration-based body temperature modifying apparatus as in any of the above embodiments, wherein the hot side heating elements reside exteriorly of the helmet and the cold side cooling elements reside in the respiration zone.

In a twenty-fourth embodiment, the present invention provides a respiration-based body temperature modifying apparatus as in any of the above embodiments, wherein the helmet is open at the neck of a user to define an outlet, with exhaled air exiting at the outlet.

In a twenty-fifth embodiment, the present invention provides a respiration-based body temperature modifying apparatus as in any of the above embodiments, wherein the air temperature modifying element is provided by a device selected from the group consisting of ceramic strip heaters, refrigerant systems, and thermoelectric heating and cooling devices.

In a twenty-sixth embodiment, the present invention provides a respiration-based body temperature modifying apparatus as in any of the above embodiments, further comprising a condensation vent through a wall in the respiration chamber to allow condensation in the respiration chamber to reach the exterior atmosphere.

In a twenty-seventh embodiment, the present invention provides a respiration-based body temperature modifying apparatus as in any of the above embodiments, further comprising a power source powering the air temperature modifying element.

In a twenty-eighth embodiment, the present invention provides a respiration-based body temperature modifying apparatus as in any of the above embodiments, wherein the power source is selected from batteries, mains power supply, solar power, auxiliary power unit, power from vehicle, and battery system integral to the apparatus's structure.

In a twenty-ninth embodiment, the present invention provides a respiration-based body temperature modifying apparatus as in any of the above embodiments, wherein the respiration zone is selected from a mask, protective face mask, protective face shield, a mask solely for device, orthopedic surgeons protective suit, non-protective mask, and other protective equipment for operators.

### BRIEF DESCRIPTION OF THE DRAWINGS

FIG. 1 is a schematic side view, in cross section showing aspects of a first embodiment of a respiration-based body temperature modifying apparatus of this invention;

FIG. 2 is a schematic side view, in cross section showing aspects of a second embodiment of a respiration-based body temperature modifying apparatus of this invention;

FIG. 3 is a schematic side view, in cross section showing aspects of a third embodiment of a respiration-based body temperature modifying apparatus of this invention;

FIG. 4 is a schematic side view, in cross section showing aspects of a fourth embodiment of a respiration-based body temperature modifying apparatus of this invention, shown particularly as part of a helmet.

### DETAILED DESCRIPTION OF ILLUSTRATIVE EMBODIMENTS

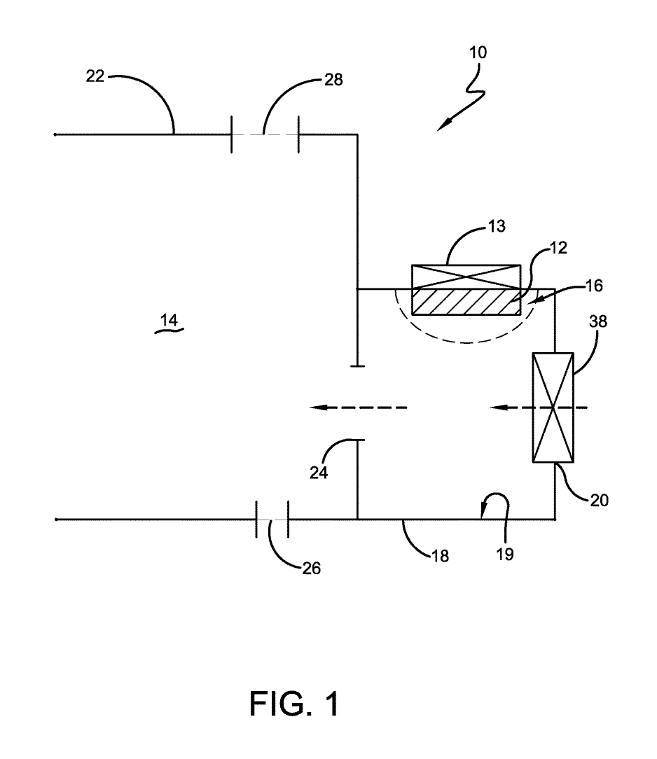

With reference to FIG. 1, the present invention provides a respiration-based body temperature modifying apparatus 10 comprising an air temperature modifying element 12 and a respiration zone 14. The air temperature modifying element 12 produces temperature-altered air and this air is then drawn in or otherwise delivered to the user's lungs. The temperature-altered air defines a respiration zone 14 that, in use, communicates with the respiratory system of a user and receives temperature-altered air so that the user can draw in the temperature-altered air from the respiration zone 14 into the lungs in order to alter body temperature. The respiration zone 14 is defined as the area proximate the user's mouth or nose or both (depending upon which is employed to inhale the temperature-altered air) and receives temperature altered air.

The apparatus 10 is intended to maintain a user's body core temperature and aid in the regulation of heart rate through a user's respiration. In some embodiments, the apparatus 10 is intended to heat a user's body core temperature. In some embodiments, the apparatus 10 is intended to cool a user's body core temperature. In some embodiments, the apparatus 10 is selectively configured to either heat or cool a user's body core temperature.

The apparatus 10 has two modes of operation to increase comfort and mitigate temperature related illnesses, depending on whether the user is in a hot or cold environment. In some embodiments, the apparatus 10 may be employed directly on a user's face, and, in other embodiments, may be employed in a manner distanced off of a user's face.

The air temperature modifying element 12 produces temperature-altered air to create the necessary temperature of breathable air for the user. In some embodiments, where there is a need to reduce the user's body core temperature (e.g., in a hot environment), cold air is supplied to the user, such that the user aspirates cold air into the lungs. In such embodiments, heat is transferred from the lungs to the cold inhaled air, and body heat is thus expelled during exhalation. In other embodiments, where there is a need to increase the user's body core temperature (e.g. in a cold environment), hot air is supplied to the user, such that the user aspirates hot air in to the lungs, and heat is transferred from the hot air into the body through the lungs. Cold air may be defined as air lower in temperature than a user's body temperature. Hot air may be defined as air higher in temperature than a user's body temperature.

In some embodiments, when cooling, a user will, upon inhaling, draw air in over the air temperature modifying element 12, and the air will be cooled and supplied to the respiration zone 14, where the user will breathe relatively cooler air into the lungs. In some embodiments, when heating, a user will breath air in over the air temperature modifying element 12, the air will be heated and supplied to the respiration zone 14, where the user will breathe relatively warmer air into the lungs.

In some embodiments, the air temperature modifying element 12 is provided by a device selected from the group consisting of ceramic strip heaters, refrigerant systems, and thermoelectric heating and cooling devices. In some embodiments, where the air temperature modifying element 12 is a ceramic strip heater, the ceramic strip heater is used to increase the user's body core temperature only. In such embodiments, the user will aspirate hot air into the lungs, and heat will be transferred from the hot air provided by the air temperature modifying element 12 into the body through the lungs.

In some embodiments, the air temperature modifying element 12 further includes a power source 13 to power the air temperature modifying element 12. In such embodiments, the power source may be selected from batteries, mains power supply, solar power, auxiliary power unit, power from vehicle, and battery system integral to the apparatus's structure.

In reference to FIG. 1, the present invention also provides an inlet zone 16. The inlet zone 16 defines the area where ambient air proximate to the air temperature modifying element 12 is affected by the air temperature modifying element 12 to produce the temperature-altered air.

In some embodiments, the apparatus 10 includes an inlet chamber 18 and an inlet passage 20, and the air temperature modifying element 12 (or at least a portion thereof) and the inlet zone 16 reside in the inlet chamber 18. The inlet passage 20 provides for external air to pass into the inlet chamber 18. In some embodiments, the inlet passage 20 may optionally include a one-way valve 38. In some embodiments, the apparatus 10 includes a fan to move the conditioned air.

In some embodiments, the inlet chamber 18 is defined by at least one wall 19 that serves to isolate the inlet chamber 18 from exterior influences on the efficiency of the temperature modification achieved through the air temperature modifying element.

In some embodiments, the respiration zone 14 resides within a respiration chamber 22, and an inhalation passage 24 allows temperature-altered air to pass from the inlet chamber 18 to the respiration chamber 22.

In some embodiments, the respiration chamber 22 optionally includes a condensation vent 26 located in a wall within the respiration chamber 22. The condensation vent 26 allows condensation build-up within the respiration chamber 22 to reach the exterior atmosphere.

In some embodiments, the respiration chamber 22 is sealed about a user's face. In such embodiments, the respiration chamber 22 will cover the user's mouth or nose or both the mouth and nose. In some such embodiments, the user's inhalation draws air in from the inlet chamber 18 through the inhalation passage 24 and into the respiration zone 14. In some such embodiments, the user's inhalation is the only driving force and draws air in at inlet 20 as well.

In some embodiments, the respiration chamber 22 further includes an outlet 28 that allows for exhausting a user's exhaled breath to the exterior atmosphere.

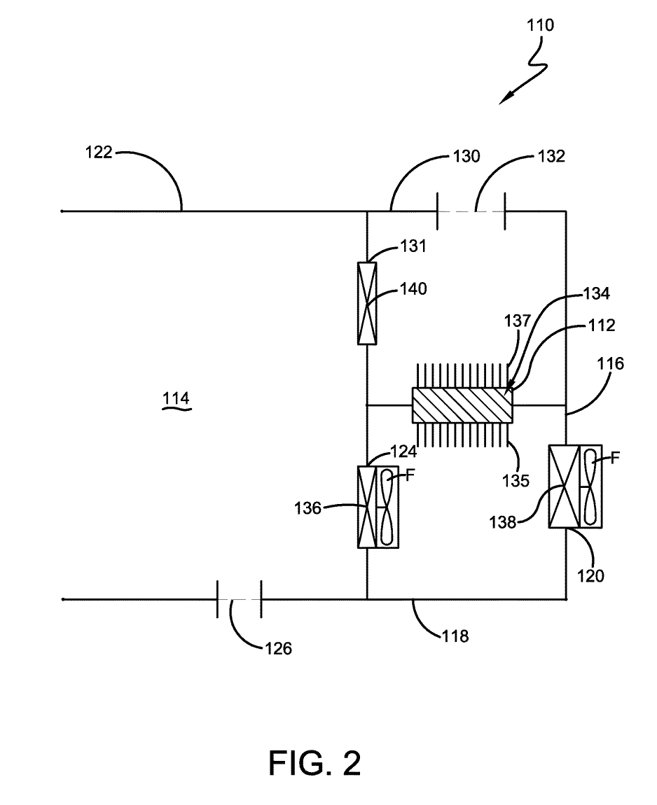

With reference to FIG. 2, the present invention may include an outlet chamber 130 and an outlet 132 in the outlet chamber 130. Like parts receive like numerals as compared to the embodiments of FIG. 1, but increased by 100. The outlet 132 in the outlet chamber 130 allows for exhausting exhaled breath of the user to the exterior atmosphere. In some embodiments, the respiration chamber 122 communicates with the outlet chamber 130 through an exhalation passage 131. In some embodiments, the exhalation passage 131 may optionally include a one-way exhalation valve 140. The one-way exhalation valve 140 can be located in either the exhalation passage 131 or in the outlet chamber 130, wherein the one-way exhalation valve 140 is chosen such that that user's respiration is sufficient to exhaust exhaled air through the one-way exhalation valve 140.

In some embodiments, the air temperature modifying element 112 is selected from hot side heating elements and cold side cooling elements provided by a thermoelectric device 134 having both a hot side heating element 135 and a cold side cooling element 137. In such embodiments, one of the hot side heating element and the cold side cooling element resides in the inlet chamber 118 and serve as the air-temperature modifying element 112, and the other of the hot side heating element and the cold side cooling element resides in the outlet chamber 130, wherein exhaled breath of the user passes over the hot side heating element or cold side cooling element residing in the outlet chamber 130.

In some embodiments, the hot side heating elements 135 and the cold side cooling elements 137 of the thermoelectric device 134 can be selectively switched to switch the positioning of the hot side heating elements and the cold side cooling elements in the inlet chamber 118 and the outlet chamber 130, thus switching the type of air, be it heated or cooled, at the inlet zone 114.

As shown by FIG. 2, in some embodiments, when heating, a user will breathe in cooler air over the hot side heating elements 135 of the thermoelectric device 134, the air will be heated and supplied to the respiration chamber 122, where the user will breathe relatively warmer air into the lungs and will exhale relatively warmer air into the outlet chamber 130 where it is expelled through the outlet in the outlet chamber 132.

In other embodiments, when cooling, a user will breathe in hot air over the cold side cooling elements 137 of the thermoelectric device 134, and the air will be cooled and supplied to the respiration chamber 122, where the user will breathe relatively cooler air into the lungs, and will further exhale relatively warmer air into the outlet chamber 130 where it is expelled through the outlet in the outlet chamber 132.

In some embodiments, the present invention does not include an outlet chamber 130. In such embodiments, one of the hot side heating elements and the cold side cooling elements reside in the inlet chamber 118 and serve as the air-temperature modifying element 112, and the other of the hot side heating elements and the cold side cooling elements are exposed to the external environment.

In some embodiments, the inhalation passage 124 may optionally include a one-way inhalation valve 136, allowing for the passage of temperature-altered air from the inlet chamber 118 to the respiration chamber 122. In some embodiments, the one-way inhalation valve 136 is chosen such that that user's respiration is sufficient to draw temperature-altered air through the one-way inhalation valve 136. In some embodiments, the present invention further includes an inlet valve 138 located in the inlet passage 120. The inlet valve 138 is chosen such that the user's respiration is sufficient to draw external air through the inlet valve 138 and into the inlet chamber 118. In some embodiments, the inhalation passage 124 optionally includes a fan, F.

In some embodiments, the present invention further includes a fan F located in the inlet passage 120. In other embodiments, the present invention further includes a fan F located in the inhalation passage 124. In some embodiments, a fan is located on the hot side heating elements 135. In some embodiments, a fan is located on cold side cooling elements 137. In some embodiments, a fan may be located between the inlet chamber 118 and the outlet chamber 130. In some embodiments, a fan is located to blow conditioned directly onto operator.

In a specific embodiment, the thermoelectric device 134 is a Peltier chip including a plurality of heat transfer fins. The heat transfer fins serve as the cold side cooling elements and a plurality of heat transfer fins serve as the hot side heating elements (fins are seen at 135 and 137, FIG. 2). In such embodiments, the thermoelectric device 134 creates a temperature difference due to its material properties when a voltage is supplied across it. In hot environments, the heat transfer fins on the hot side are isolated and will be exposed to the outside environment or in the outlet chamber 130, and the cold side is isolated and will be kept cold within the inlet chamber 118. In a cold environment, the polarity of the thermoelectric device 134 is reversed and the hot air is supplied to the user. In cold environments, the heat transfer fins located on the cold side are isolated and will be exposed to the outside environment or in the outlet chamber 130 and the hot side is isolated and will be kept hot within the inlet chamber 118. In some embodiments, by breathing directly over the opposite side of the Peltier chip, the convention heat transfer coefficient is increased. This increase in heat transfer promotes a lower temperature of the “hot side”, thereby lowering the temperature of the “cold side.”

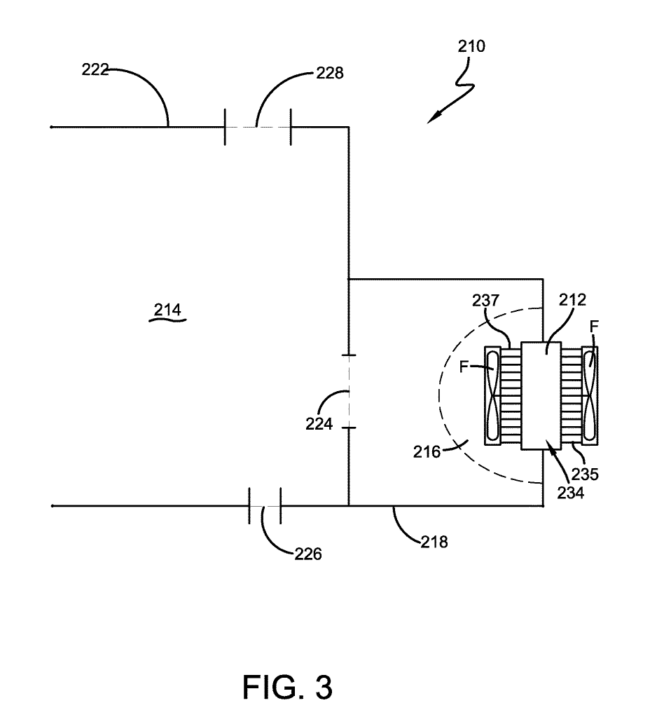

With reference to FIG. 3, another embodiment of a respiration-based body temperature modifying apparatus 210 is shown in a manner not sealed to a user's face. Like parts receive like numerals as compared to the embodiments of FIG. 2, but increased by 100. Thus, the air temperature modifying element 212 produces temperature-altered air to create the necessary temperature of breathable air for the user. In some embodiments, where there is a need to reduce the user's body core temperature (e.g. in a hot environment), cold air is supplied to the user, such that when in use, the user aspirates cold air into the lungs and facilitates heat transfer directly from the core of the body. In such embodiments, heat is transferred from the lungs by the cold air and expelled during exhalation.

In some embodiments, the respiration-based body temperature modifying apparatus 210 is employed off of a user's face. In some embodiments, the present invention provides a respiration-based body temperature modifying apparatus 210 comprising an air temperature modifying element 212 and a respiration zone 214. The air temperature modifying element 212 produces temperature-altered air and this air is delivered to the user's lungs. The temperature-altered air defines a respiration zone 214, in use, communicates with the respiratory system of a user and receives temperature-altered air so that the user inhale the temperature-altered air from the respiration zone 214 into the lungs in order to alter body temperature. In some embodiments, the respiration zone 214 can be defined as the localized area surrounding the user's face or head, or both.

The air temperature modifying element 212 produces temperature-altered air to create the necessary temperature of breathable air for the user. When cooling, a user will, upon inhaling, inhale air transferred over the air temperature modifying element 212, and the air will be cooled and supplied to the respiration zone 214, where the user will breathe relatively cooler air into the lungs. When heating, a user will inhale air transferred over the air temperature modifying element 212, the air will be heated and supplied to the respiration zone 214, where the user will breathe relatively warmer air into the lungs. In such embodiments, the respiration-based body temperature modifying apparatus 210 requires a fan, F, to draw the air over the air temperature modifying element 212 since the apparatus is off of the user's face and the user's breath is no longer applicable to draw in air.

In some embodiments, the air temperature modifying element 212 is provided by a device selected from the group consisting of ceramic strip heaters, refrigerant systems, and thermoelectric heating and cooling devices. In some embodiments, where the air temperature modifying element 212 is a ceramic strip heater, the ceramic strip heater is used to increase the user's body core temperature only. In such embodiments, the user will aspirate hot air into the lungs, and heat will be transferred from the hot air provided by the air temperature modifying element 212 into the body through the lungs.

As shown in FIG. 3, the present invention also provides an inlet zone 216. The inlet zone 216 defines the area where ambient air proximate to the air temperature modifying element 212 is affected by the air temperature modifying element 212 to produce the temperature-altered air. In some embodiments, the inlet zone 216 further provides an inlet chamber 218. The air temperature modifying element 212 (or at least a portion thereof) and the inlet zone 216 reside in the inlet chamber 218.

In some embodiments, the respiration zone 214 resides within a respiration chamber 222, and an inhalation passage 224 allows temperature-altered air to pass from the inlet chamber 218 to the respiration chamber 222. In some embodiments, the respiration chamber 222 optionally includes a condensation vent 226 located in a wall within the respiration chamber 222. The condensation vent 226 allows condensation build-up within the respiration chamber 222 to reach the exterior atmosphere. In some embodiments, the respiration chamber 222 further includes an outlet 228 that allows for exhausting a user's exhaled breath to the exterior atmosphere. In some embodiments, the respiration chamber 222 is proximate to a user's face. In such embodiments, the respiration chamber 222 will surround the user's face.

In some embodiments, the air temperature modifying element 212 is selected from hot side heating elements and cold side cooling elements provided by a thermoelectric device 234 having both hot side heating elements 235 and cold side cooling elements 237. In such embodiments, one of the hot side heating elements 235 and the cold side cooling elements 237 reside in the inlet chamber 218 and serve as the air-temperature modifying element 212, and the other of the hot side heating elements 235 and the cold side cooling elements 237 reside in the external environment.

In some embodiments, the hot side heating elements 235 and the cold side cooling elements 237 of the thermoelectric device 234 can be selectively switched to switch the positioning of the hot side heating elements and the cold side cooling elements in the inlet chamber 218 and the external environment, thus switching the type of air, be it heated or cooled, at the inlet zone 214.

In a specific embodiment, the thermoelectric device 234 is a Peltier chip including a plurality of heat transfer fins. The heat transfer fins serve as the cold side cooling elements 237 and a plurality of heat transfer fins serve as the hot side heating elements 235. In such embodiments, the thermoelectric device 234 creates a temperature difference due to its material properties when a voltage is supplied across it. In hot environments, the heat transfer fins on the hot side are isolated and will be exposed to the outside environment and the cold side is isolated and will be kept cold within the inlet chamber 218. In a cold environment, the polarity of the thermoelectric device 234 is reversed and the hot air is supplied to the user. In cold environments, the heat transfer fins located on the cold side are isolated and will be exposed to the outside environment and the hot side is isolated and will be kept hot within the inlet chamber 218.

In some embodiments, the respiration-based body temperature modifying apparatus 210 may be employed within a helmet, a hazmat suit, protective face mask, protective face shield, a mask solely for device, orthopedic surgeons protective suit, non-protective mask, or other protective equipment for operators.

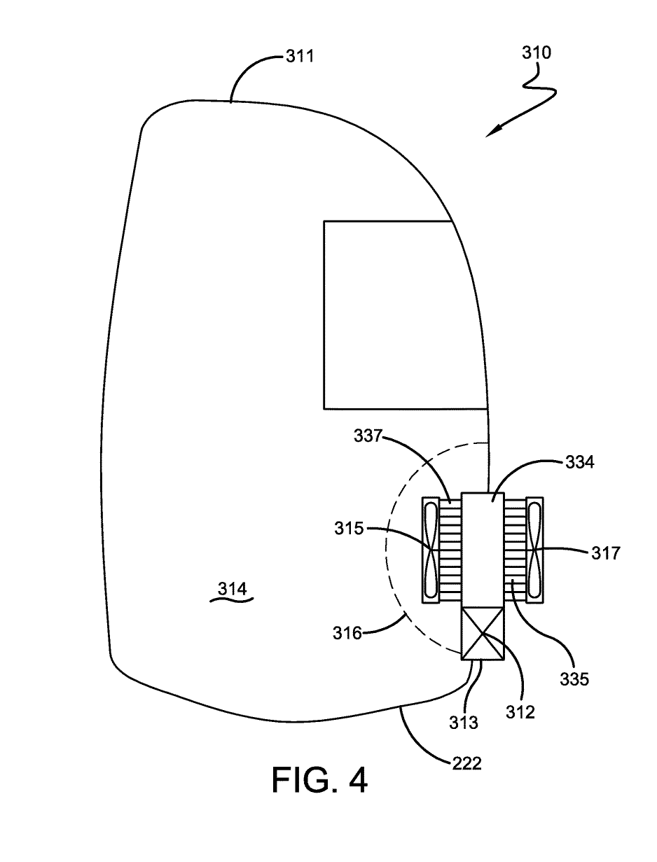

With reference to FIG. 4, in a specific embodiment, the respiration-based body temperature modifying apparatus 310 can be implemented within a helmet 311, wherein the respiration zone 314 is provided therein. Like parts receive like numerals as compared to the embodiments of FIG. 3, but increased by 100. In some embodiments, the helmet 311 is open at the neck of a user to define an outlet, where exhaled air exits through the outlet.

In some embodiments, the air temperature modifying element 312 is selected from hot side heating elements 335 and cold side cooling elements 337 provided by a thermoelectric device 334 having both hot side heating elements 335 and cold side cooling elements 337, such that one of hot side heating elements and the cold side cooling elements reside in the respiration zone 314 and serve as the air-temperature modifying element 312. In some embodiments, the respiration zone 314 further comprises respiration side fan 315. The respiration side fan 315 moves air across the hot side heating or cold side cooling elements residing on the respiration zone 314.

In some embodiments, the other of the hot side heating elements 335 and the cold side cooling elements 337 resides externally of the helmet 311. In such embodiments, the air temperature modifying element 312 further comprises a fan 317 moving air across the hot side heating elements 335 or cold side cooling elements 337 residing exteriorly of the helmet 311. In such embodiments, the hot side heating elements 335 and the cold side cooling elements 337 can be selectively switched to switch the positioning of the hot side heating elements 335 and the cold side cooling elements 337 in the respiration zone 314 and exteriorly of the helmet 311.

In a specific embodiment, the air temperature modifying element 312 is a Peltier chip. The Peltier chip includes a plurality of heat transfer fins serving as the cold side cooling elements 337 and a plurality of heat transfer fins serving as the hot side heating elements 335. With reference to FIG. 4, in some embodiments, the hot side heating elements 335 reside exteriorly of the helmet 311 and the cold side cooling elements 337 reside in the respiration zone 314. In other embodiments, the cold side cooling elements 337 reside exteriorly of the helmet 311 and the hot side heating elements 335 reside in the respiration zone 314.

In some embodiments, the air temperature modifying element 312 further includes a power source 313 to power the air temperature modifying element 312. In such embodiments, the power source may be selected from batteries, mains power supply, solar power, auxiliary power unit, power from vehicle, and battery system integral to the apparatus's structure.

In light of the foregoing, it should be appreciated that the present invention significantly advances the art by providing a respiration-based body temperature modifying apparatus that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

What is claimed is:

1. A respiration-based body temperature modifying apparatus comprising:
an air temperature modifying element producing temperature-altered air; and
a respiration zone that, in use, communicates with the respiratory system of the user and receives said temperature-altered air so that the user can draw the temperature-altered air from the respiration zone into the lungs to alter body temperature.

2. The apparatus of claim 1, including an inlet zone defined where ambient air proximate the air temperature modifying element is affected by the air temperature modifying element to produce the temperature-altered air.

3. The apparatus of claim 2, further comprising:
an inlet chamber, wherein said air temperature modifying element and said inlet zone reside in said inlet chamber; and
an inlet passage for the passage of external air into said inlet chamber.

4. The apparatus of claim 3, further comprising:
a respiration chamber, said respiration zone residing in said respiration chamber; and
a inhalation passage from said inlet chamber to said respiration chamber for the passage of temperature-altered air from said inlet chamber to said respiration chamber.

5. The apparatus of claim 4, wherein said respiration chamber, in use, seals about the user's face to cover a mouth or nose or both the mouth and nose of the user.

6. The apparatus of claim 5, further comprising:
an outlet in said respiration chamber for exhausting exhaled breath of the user to the exterior atmosphere.

7. The apparatus of claim 5, further comprising:
an outlet chamber; and
an outlet in said outlet chamber for exhausting exhaled breath of the user to the exterior atmosphere, said respiration chamber communicating with the outlet chamber through an exhalation passage.

8. The apparatus of claim 7, wherein said air temperature modifying element is selected from hot side heating elements and cold side cooling elements provided by a thermoelectric device having both said hot side heating elements and said cold side cooling elements, such that one of said hot side heating elements and said cold side cooling elements reside in said inlet chamber and serve as said air-temperature modifying element, and the other of said hot side heating elements and said cold side cooling elements reside in said outlet chamber, wherein exhaled breath of the user passes over the hot side heating or cold side cooling element residing in said outlet chamber.

9. The apparatus of claim 8, further comprising a one-way inhalation valve in said inhalation passage allowing for the passage of temperature-altered air from said inlet chamber to said respiration chamber.

10. The apparatus of claim 9, wherein the one-way inhalation valve is chosen such that the user's respiration is sufficient to draw temperature-altered air through the one-way inhalation valve.

11. The apparatus of claim 10, further comprising an inlet valve in said inlet passage, wherein said inlet valve is chosen such that the user's respiration is sufficient to draw external air through said inlet valve and into said inlet chamber.

12. The apparatus of claim 8, wherein said hot side heating elements and said cold side cooling elements can be selectively switched to switch the positioning of the hot side heating elements and the cold side cooling elements in said inlet chamber and said outlet chamber, thus switching the type of air, be it heated or cooled, at the inlet zone.

13. The apparatus of claim 12, wherein said thermoelectric device is a Peltier chip including a plurality of heat transfer fins serving as said cold side cooling elements and a plurality of heat transfer fins serving as said hot side heating elements.

14. The apparatus of claim 1, further comprising a helmet, wherein said respiration zone is provided in said helmet.

15. The apparatus of claim 14, wherein said air temperature modifying element is selected from hot side heating elements and cold side cooling elements provided by a thermoelectric device having both said hot side heating elements and said cold side cooling elements, such that one of hot side heating elements and said cold side cooling elements reside in said respiration zone and serve as said air-temperature modifying element.

16. The apparatus of claim 15, wherein the other of said hot side heating elements and said cold side cooling elements reside exteriorly of the helmet.

17. The apparatus of claim 16, further comprising a fan moving air across said other of said hot side heating elements or cold side cooling elements residing exteriorly of the helmet.

18. The apparatus of claim 16, wherein said hot side heating elements and said cold side cooling elements can be selectively switched to switch the positioning of said hot side heating elements and said cold side cooling elements in said respiration zone and exteriorly of said helmet.

19. The apparatus of claim 18, wherein said air temperature modifying element is a Peltier chip including a plurality of heat transfer fins serving as said cold side cooling elements and a plurality of heat transfer fins serving as said hot side heating elements.

20. The apparatus of claim 15, wherein the helmet is open at the neck of a user to define an outlet, with exhaled air exiting at said outlet.
